(19) United States
(12) Patent Application Publication
(10) Pub. No.: US 2024/0099982 A1
(43) Pub. Date: Mar. 28, 2024

(54) CELLULOSE DERIVATIVE FREE CAPSULE FOR HERBAL EXTRACTS

(71) Applicant: GAIA HERBS, Brevard, NC (US)

(72) Inventors: David Jeremy Stewart, Brevard, NC (US); Ido D. Dagan, Asheville, NC (US); Greg Masters, Brevard, NC (US)

(21) Appl. No.: 18/372,209

(22) Filed: Sep. 25, 2023

Related U.S. Application Data
(60) Provisional application No. 63/410,278, filed on Sep. 27, 2022.

(51) Int. Cl. A61K 9/48; A61K 36/41; A61K 36/53; A61K 36/79; A61K 36/81; A61K 36/9066

(52) U.S. Cl. CPC A61K 9/4816; A61K 9/4858; A61K 9/4875; A61K 36/41; A61K 36/53; A61K 36/79; A61K 36/81; A61K 36/9066; A61K 2236/333; A61K 2236/37

(57) ABSTRACT

The present invention provides a cellulose derivative free capsule that may be organic and plant-based. The capsule includes organic pullulan, one or more herbal extracts in a powder form or as an aqueous alcoholic extract, and a mixture of plant-based oil and plant-based lecithin. The mixture of oil and lecithin may have a viscosity of 250 to 1000 cP at 25° C., and with the herbal extract may have a viscosity of 1000 to 2500 cP at 25° C.

### CROSS-REFERENCE TO RELATED APPLICATION

This application claims the benefit of priority to U.S. Provisional Application Ser. No. 63/410,278 filed Sep. 27, 2022.

### FIELD OF THE INVENTION

The present invention relates to a capsule for herbal extracts which is devoid of cellulose derivatives.

### BACKGROUND

Herbal extracts in powder or liquid form are a popular source of medical and nutritional support. Herbal remedies, sometimes referred to as nutraceuticals or dietary supplements are alternatives or adjuncts to conventional pharmaceutical drugs. Various formulations for capsules and tables for herbal contracts are known. For example, cellulose derivative capsules based on hydroxypropyl methylcellulose (HPMC) are known. One example is described in U.S. Pat. No. 6,238,696 B1 to Wang, et al, in which liquid extracts are provided in an HPMC or gelatin capsule. HPMC and gelatin are, however, not typically organically derived.

Thus it would be desirable to provide a capsule or the like for the delivery of an herbal extract or herbal extracts in a powder or liquid form that is devoid of cellulose derived materials and utilizes plant-based components.

### SUMMARY

The present invention provides a cellulose derivative free capsule that may be organic and plant-based. The capsule includes organic pullulan, one or more herbal extracts in powder form or as an aqueous alcoholic extract, and a mixture of plant-based oil and plant-based lecithin. The mixture of oil and lecithin may have a viscosity of 250 to 1000 cP at 25° C., and with the herbal extract may have a viscosity of 1000 to 2500 cP at 25° C.

### DETAILED DESCRIPTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. Unless otherwise defined, all terms (including technical and scientific terms) used herein have the same meaning as commonly understood by one having ordinary skill in the art to which this invention belongs.

It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

All patents, patent applications and publications referred to herein are incorporated by reference in their entirety. In case of a conflict in terminology, the present specification is controlling.

In the following description, for purposes of explanation, numerous specific details are set forth in order to provide a thorough understanding of the present invention. It will be evident, however, to one skilled in the art that the present invention may be practiced without these specific details. The present disclosure is to be considered as an exemplification of the invention and is not intended to limit the invention to the specific embodiments illustrated by the figures or description below. As is well known to those skilled in the art, many careful considerations and compromises typically must be made when designing for the optimal configuration of a commercial implementation of any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

As discussed above, an organic pullulan capsule encapsulating an herbal extract is provided. The capsule may include one or more herbal extracts. The extracts may be in powder form or as an aqueous alcohol extract. The capsule may also include a mixture of a plant-based oil(s) and plant-based lecithin(s). Suitable additional excipients may be added to the extract prior to encapsulation, and may include waxes (e.g., carnauba), fats, semi-solid and liquid polyols and the like.

The capsule may be a two-piece capsule manufactured utilizing conventional encapsulation equipment such as the encapsulators available from Shinogi. In one embodiment, the encapsulation may include removal of air using an inert gas such as nitrogen.

By utilizing organic pullulan, a capsule devoid of cellulose derivatives may be provided. “Devoid” of cellulose derivatives or “cellulose derivative-free” is intended to mean less than 1 percent, often less than 0.5 percent, and preferably 0 percent presence of cellulose derivatives.

The herbal extracts may be derived from herbal material in the form of whole leaf, stem, stalk, root and the like, and may be cut or ground prior to use or to extraction. The herbal material may include, but not limited to one or more of kava, echinacea, St. John's wort, valerian root, milk thistle seed, Siberian ginseng, nettle leaf, holy basil, ginkgo, gotu kola, astragalus, goldenseal, dong quai, ginseng, bilberry, green tea, hawthorne, ginger, turmeric, Rhodiola rosea, black cohosh, cats claw, chamomile, dandelion, chaste tree berry, feverfew, garlic, horse chestnut, licorice, eyebright, yohimbe, valerian poppy, black pepper, arnica, black elderberry, ashwagandha, acacia, hemp, acai, allspice, skullcap, burdock and maca and various other herbal materials listed, for example in the Material Medica of Western Herbs by C. Fisher or Homoeopathic Materia Medica by H. Boericke.

The extracts may be in solid powder form or may be extracted in one embodiment with an aqueous alcohol in different concentrations to provide an aqueous alcoholic herbal extract. Suitable alcohols may include C1 to C3 alcohols like ethanol. Preferably, ethanol may be used. The alcohol may be a co-solvent mixture such as a mixture of alcohol and water. For example, the alcohol mixture may be an ethanol solution ranging from about 70% w/v to about 90% w/v. In a preferred embodiment, a 70% w/v ethanol solution may be used to extract the herbal material. During extraction, the herbal material is preferably percolated or macerated to facilitate extraction. In another embodiment, supercritical extraction (e.g. with CO₂) may be utilized instead of solvent extraction to extract the herbal material.

The capsule portion may preferably be organic pullulan. Pullulan is a natural water soluble polysaccharide. Specifically, it is a polysaccharide polymer of a malttriose trimer made up of α-1,4-α-1,6-glucan. Pullulan is an organic polymer and may be produced from starch by the fungus Auerobasidium pullulans. A method of production is described, for example, in U.S. Pat. No. 10,894,971 B1. Pullulan functions as an oxygen barrier, disintegrates rapidly, and is stable. It is noted that pullulan is soluble in glycerin, thus use of glycerin should be avoided. A source of organic pullulan is Bright Pharma Caps.

A lecithin/oil mixture may be included in the capsule and may function as an emulsifier. The lecithin and oil may be both plant based, and preferably are organic. Suitable plant-based lecithin sources may include corn lecithin, cottonseed oil lecithin, rapeseed lecithin, soybean lecithin and sunflower lecithin. Suitable plant-based oils may be oils derived from corn, cottonseed, flaxseed, grapeseed, hemp oil, nuts (e.g., almond and peanut), olives, sunflower, soybean, safflower and sesame seed and the like.

The lecithin/oil mixture contributes to the overall viscosity. The viscosity of the oil may be about 30 to 70 cP at 25° C. and the viscosity of the lecithin may be greater than about 12,000 cP at 25° C. The oil and lecithin mixture may have a viscosity of 250 to 1000 cP at 25 ° C. The ratio of plant based lecithin to plant based oil in one embodiment is about 4:1 to about 1:4. In another embodiment only plant-based lecithin may be used. The overall mixture including the herbal extract may have a viscosity of about 1000-2500 cP at 25° C.

The organic pullulan capsule may comprise about 5 to 40 percent herbal extract, and about 70 to 97 percent plant-based oil and plant-based lecithin mixture. The capsule may also include about 0 to 10 percent excipients such as carnauba wax. The two-piece capsules may be sealed using a sealant composition comprising about 10 to 25 percent by weight organic pullulan, 20 to 35 percent by weight of a short-chained alcohol such as ethanol and 50 to 75 percent by weight deionized water. In this manner, leakage of the extract, particularly in liquid form may be avoided if a two-piece capsule is utilized.

The following examples are merely illustrative of the inventions and formulations thereof.

### EXAMPLES

### Example 1 Oil of Oregano

A capsule formed from organic pullulan from Bright Pharma Caps includes 15 percent by weight supercritical oregano leaf and 85 percent by weight of a 95/5 plant-based lecithin/olive oil mixture.

### Example 2 Adrenal Health

A capsule formed from organic pullulan from Bright Pharma Caps is filled with 20 percent by weight hydroethanolic extract of holy basil, schisandra berry and wild oats, 9 percent by weight Rhodiola rosea powdered extract, 7 percent by weight ashwagandha powdered extract, 2 percent by weight holy basil supercritical extract, and 62 percent by weight of a 50/50 plant-based lecithin/olive oil mixture.

### Example 3 Ashwagandha

A capsule formed from organic pullulan from Bright Pharma Caps is filled with 10 percent by weight hydroethanolic extract of ashwagandha, 23 percent by weight ashwagandha powdered extract, and 67 percent by weight of a 50/50 plant-based lecithin/olive oil mixture.

### Example 4 Vitex™ (Chaste Tree)

A capsule formed from organic pullulan from Bright Pharma Caps is filled with 35 percent by weight hydroethanolic extract of chaste tree berries, and 65 percent by weight of a 75/25 plant-based lecithin/olive oil mixture.

### Example 5 Turmeric

A capsule formed from organic pullulan from Bright Pharma Caps is filled with 30 percent by weight hydroethanolic extract of turmeric, 8 percent by weight curcuminoid powdered extract, 5 percent by weight turmeric supercritical extract, 1 percent by weight black pepper supercritical extract, and 56 percent by weight of a 75/25 plant-based lecithin/olive oil mixture.

### Example 6 Rhodiola Rosea

A capsule formed from organic pullulan from Bright Pharma caps is filled with 20 percent by weight hydroethanolic extract of Rhodiola rosea and 80 percent by weight of an 87.5/12.5 plant-based lecithin/olive oil mixture. The two-piece capsule is sealed with a sealant comprising 16 percent by weight organic pullulan powder, 28 percent by weight ethanol and 56 percent by weight deionized water.

Although the present approach has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present approach.

That which is claimed is:

1. An organic or plant-based cellulose derivative free capsule encapsulating an herbal extract, the capsule comprising:
a. organic pullulan;
b. one or more herbal extracts as a powder or as an aqueous alcoholic extract, and
c. a mixture of plant-based oil and a plant-based lecithin, wherein the viscosity of the one or more herbal extracts and mixture of plant-based oil and plant-based lecithin has a viscosity of about 1000 to 2500 cP at 25° C.

2. The organic and plant-based cellulose derivative free capsule of claim 1, wherein the ratio of plant-based oil to plant-based lecithin is about 4:1 to about 1:4.

3. The organic and plant-based cellulose derivative free capsule of claim 1 further comprising an excipient.

4. The organic and plant-based cellulose derivative free capsule of claim 3, wherein the excipient is carnauba wax.

5. The organic and plant-based cellulose derivative free capsule of claim 1, wherein the one or more herbal extract is derived from a herbal plant material selected from the group consisting of kava, echinacea, St. John's wort, valerian root, milk thistle seed, Siberian ginseng, nettle leaf, holy basil, ginkgo, gotu kola, astragalus, goldenseal, dong quai, ginseng, bilberry, green tea, hawthorne, ginger, turmeric, Rhodiola rosea, black cohosh, cats claw, chamomile, dandelion, chaste tree berry, feverfew, garlic, horse chestnut, licorice, eyebright, yohimbe, valerian poppy, black pepper, arnica, black elderberry, ashwagandha, acacia, hemp, acai, allspice, skullcap, burdock and maca.

6. The organic and plant-based cellulose derivative free capsule of claim 1, wherein the herbal material is extracted using supercritical extraction.

7. A two-piece capsule encapsulating an herbal extract, the capsule being organic or plant-based cellulose derivative free, the two-piece capsule comprising organic pullulan as the capsule material, about 5 to 45 percent by weight of one or more herbal extracts in powder form or as an aqueous alcohol extract and about 40 to 97 percent by weight of a mixture of plant-based oil and plant-based lecithin in the capsule, and a sealant sealing the two-piece capsule together, the sealant comprising about 10 to 25 percent by weight organic pullulan, 20 to 35 percent by weight of a short-chained alcohol, and 50 to 75 percent by weight deionized water.

8. The organic and plant-based cellulose derivative free capsule of claim 7, wherein the ratio of plant-based oil to plant-based lecithin is about 4:1 to about 1:4.

9. The organic and plant-based cellulose derivative free capsule of claim 7 further comprising an excipient.

10. The organic and plant-based cellulose derivative free capsule of claim 9, wherein the excipient is carnauba wax.

11. The organic and plant-based cellulose derivative free capsule of claim 7, wherein the one or more herbal extract is derived from a herbal plant material selected from the group consisting of kava, echinacea, St. John's wort, valerian root, milk thistle seed, Siberian ginseng, nettle leaf, holy basil, ginkgo, gotu kola, astragalus, goldenseal, dong quai, ginseng, bilberry, green tea, hawthorne, ginger, turmeric, Rhodiola rosea, black cohosh, cats claw, chamomile, dandelion, chaste tree berry, feverfew, garlic, horse chestnut, licorice, eyebright, yohimbe, valerian poppy, black pepper, arnica, black elderberry, ashwagandha, acacia, hemp, acai, allspice, skullcap, burdock and maca.

12. The organic and plant-based cellulose derivative free capsule of claim 7, wherein the herbal material is extracted using supercritical extraction.

13. The organic and plant-based cellulose derivative free capsule of claim 7, wherein the short-chained alcohol is ethanol.
